(19) United States
(12) Patent Application Publication
(10) Pub. No.: US 2024/0366487 A1
(43) Pub. Date: Nov. 7, 2024

(54) ANTI-AGING COMPOSITIONS FOR SENSITIVE SKIN

(71) Applicant: Baek Clinical Inc., Los Angeles, CA (US)

(72) Inventor: Jihoon P. Baek, Los Angeles, CA (US)

(21) Appl. No.: 18/653,891

(22) Filed: May 2, 2024

Related U.S. Application Data
(60) Provisional application No. 63/499,698, filed on May 2, 2023.

(51) Int. Cl. A61K 8/49; A45D 40/00; A61K 8/34; A61K 8/36; A61K 8/368; A61K 8/44; A61K 8/55; A61K 8/60; A61K 8/9767; A61Q 19/08

(52) U.S. Cl. CPC A61K 8/4926; A45D 40/00; A61K 8/345; A61K 8/36; A61K 8/368; A61K 8/44; A61K 8/55; A61K 8/602; A61K 8/9767; A61Q 19/08

(57) ABSTRACT

Aspects of the present disclosure include compositions and methods for treating, preventing, or improving dermatocosmetic conditions, the composition comprising topical formulations containing a combination of niacinamide and galactoarabinan.

### 1. CROSS-REFERENCE TO RELATED APPLICATIONS

This application claims the benefit of U.S. Provisional Application No. 63/499,698, filed May 2, 2023, which is hereby incorporated in its entirety by reference herein.

### 2. INTRODUCTION

Uneven skin texture, skin mottling and uneven skin tone, fine wrinkling, and skin dryness are hallmarks of chronological aging and skin damaged by sun exposure. Prudent sun protective practices, combined with topical cosmetic and dermatological treatments can improve the appearance of such hallmarks of chronologically aged and photodamaged skin, but are at times poorly tolerated by individuals who perceive their skin type as being sensitive. In such individuals, sensations of stinging and burning, along with signs of irritation, such as skin redness and dryness, can be obstacles to continued and consistent product usage, which are required for long-term improvements. Ingredients that are often associated with such challenges include, but are not limited to, retinol and other retinoids, glycolic acid, and ascorbic acid.

Niacinamide, also known as nicotinamide, is known to be relatively well-tolerated even in populations who identify themselves as having sensitive skin, and with consistent and continued usage, is known to improve a number of characteristics associated with photoaged skin, including, but not limited to, uneven skin tone, fine wrinkling, and impaired barrier function. Although generally considered to be gentle, because the visible anti-photoaging effects of niacinamide are less dramatic than other commonly used compounds, such as retinol and glycolic acid, it benefits from being combined with other compounds that can maximize its effects on skin.

Galactoarabinan, also known as arabinogalactan and larch gum, is one potential compound. Galactoarabinan is a polysaccharide that occurs in the primary and secondary cell walls of plant cells, and commercially sourced from North American larch trees. It has been the subject of study with respect to its effects on human health, with evidence demonstrating its effects on gut microflora the immune system when ingested. In topical applications, it is sometimes used as an agent to help products form an even and adherent layer to skin, support skin hydration, and to help disperse pigment particles, and is not known to be a common skin irritant or sensitizer.

There has been and remains a need for topical formulations that significantly improve multiple signs of chronological aging and photodamage while minimizing the adverse effects often associated with such improvements, such as visible and palpable signs of irritation and dryness.

### 3. SUMMARY

Aspects of the present disclosure include compositions and methods for treating, preventing, or improving dermatocosmetic conditions.

Topical formulations containing a combination of niacinamide and galactoarabinan. The formulations contain niacinamide and galactoarabinan and have desirable physical properties. The topical formulations can include concentrations of niacinamide of 1% to 20% by weight, and galactoarabinan of 0.1% to 10% by weight. The topical compositions of this disclosure find use in treating or preventing a variety of cosmetic and/or dermatological conditions as well as reducing the appearance of chronological and/or environmentally-caused skin aging.

The present inventor surprisingly found that there was a synergistic effect of niacinamide and galactoarabinan when combined in a skincare formulation described herein, where galactoarabinan was surprisingly good at exfoliating the skin at a moderate pH (e.g pH of 4 to 7). Unlike compounds such as N-Acetylglucosamine which, when combined with niacinamide, augment the properties of niacinamide, galactoarabinan, which has exfoliating properties and is pH independent, enhances the properties of niacinamide. The present inventor also found that the formulation, with a low pH (e.g. 4 to 7), minimizes chances of the byproduct of niacin causing flushing or redness of the skin.

### 4. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 provides Baseline (left photo) photography taken via Visia-CR device and shows fine wrinkling in the periocular region, as well as hyperpigmentation and uneven skin tone across the cheeks and forehead. After 8 weeks (right photo), fine wrinkling, uneven skin tone, and hyperpigmentation are visibly improved.

### 5. DETAILED DESCRIPTION OF THE INVENTION

This disclosure provides topical formulations comprising niacinamide and galactoarabinan. The formulations provide improvements in skin wrinkling, uneven skin tone, and hydration. This disclosure provides particular topical formulations which have been developed and optimized to provide skin compatibility and desirable physical properties.

Topical compositions of this disclosure find use in treating or preventing a variety of cosmetic and/or dermatological conditions as well as to reduce the appearance of chronological and/or environmentally-caused skin aging, such as facial fine lines and wrinkles, dyschromia or uneven pigmentation, and uneven skin texture. Non-limiting examples of dermatocosmetic conditions that may be improved by topical application of the compositions of the present disclosure include: keratoses, melasma, lentigines, liver spots, inflammatory dermatoses (including eczema, acne, psoriasis), and xeroses (also known in the art as dry skin or pruritus).

In some embodiments, formulations of the present disclosure include the ingredients: (i) 1% to 20% by weight niacinamide; (ii).1% to 10% by weight galactoarabinan dissolved in (iii) an aqueous system with (iii) a pH between 2.5 and 7.0.

### 5.1. Niacinamide

Niacinamide comprises the formula C6H6N2O. Niacinamide, also known as nicotinamide, is known to be relatively well-tolerated even in populations who identify themselves as having sensitive skin, and with consistent and continued usage, is known to improve a number of characteristics associated with photoaged skin, including, but not limited to, uneven skin tone, fine wrinkling, and impaired barrier function. Although generally considered to be gentle, because the visible anti-photoaging effects of niacinamide are less dramatic than other commonly used compounds, such as retinol and glycolic acid, it benefits from being combined with other compounds that can maximize its effects on skin.

Niacinamide or nicotinamide is similar to niacin, and is critical vitamins to a number of important functions, including the NAD/NADP complex. NAD is critical to catabolismof fat, carbohydrate, protein, and alcohol and cell signaling and DNA repair. NADP is important for anabolic reactions in fatty acid and cholesterol synthesis. Its use in the compositions along with niacin is to provide these required compounds that are important in these types of reactions.

Vitamin B3 (VTB3) or Niacin comprises the Formula C6H5NO2. Niacin cannot be directly converted to niacinamide, but both are critical precursors of the important NAD/NADP complex. NAD is critical to catabolismof fat, carbohydrate, protein, alcohol, cell signaling and DNA repair. NADP is important for anabolic reactions in fatty acid and cholesterol synthesis. Niacin also is a vasodilator that can also aid in the absorption of the composition on the skin. Their uses in the compositions along with niacinamide are to provide these required compounds that are important in these reactions.

Compositions of the present disclosure comprise niacinamide. In some embodiments, the composition comprises 0.5 to 20% by weight of niacinamide. In some embodiments, composition includes about 1 to 20% by weight (e.g. about 1% or more, about 1.5% or more, about 2% or more, about 2.5% or more, about 3% or more, about 3.5% or more, about 4% or more, about 4.5% or more, about 5% or more, about 5.5% or more, about 6% or more, about 7% or more, about 7.5% or more, about 8% or more, about 8.5% or more, about 9% or more, about 10% or more, about 10.5% or more, about 11% or more, about 11.5% or more, about 12% or more, about 12.5% or more, about 13% or more, about 13.5% or more, about 14% or more, about 14.5% or more, about 15% or more, about 15.5% or more, about 16% or more, about 16.5% or more, about 17% or more, about 17.5% or more, about 18% or more, about 18.5% or more, about 19% or more, about 19.5% or more, or about 20% or more) of niacinamide.

### 5.2. Galactoarabinan

Galactoarabinan used interchangeably herein as “arabinogalactan”, “larch arabinogalactan”, or “larch gum” is a biopolymer that includes arabinose and galactose monosaccharides.

Galactoarabinan is a polysaccharide that occurs in the primary and secondary cell walls of plant cells, and commercially sourced from North American larch trees. It has been the subject of study with respect to its effects on human health, with evidence demonstrating its effects on gut microflora the immune system when ingested. In topical applications, it is sometimes used as an agent to help products form an even and adherent layer to skin, support skin hydration, and to help disperse pigment particles, and is not known to be a common skin irritant or sensitizer.

Compositions of the present disclosure comprise Galactoarabinan. In some embodiments, the composition comprises 0.01 to 10% by weight of Galactoarabinan. In some embodiments, composition includes about 0.01 to 10% by weight (e.g. about 0.1% or more, 0.05% or more, 0.1% or more, 0.5% or more, 1% or more, about 1.5% or more, about 2% or more, about 2.5% or more, about 3% or more, about 3.5% or more, about 4% or more, about 4.5% or more, about 5% or more, about 5.5% or more, about 6% or more, about 7% or more, about 7.5% or more, about 8% or more, about 8.5% or more, about 9% or more, or about 10% or more) of Galactoarabinan.

### 5.3. Non-Aqueous Solvents

A non-aqueous solvent of the present compositions is a solvent that does not cause irritation or sensitization when applied topically to the skin. Non-aqueous solvents of interest include polyols, C(1-6) alkanediols, glycol ethers, dimethyl ethers, and combinations thereof.

In some embodiments, the non-aqueous solvent is a skin compatible polyol. A polyol is an organic alcohol solvent having two or more hydroxy groups. In some embodiments, the polyol solvent is a C(3-6) polyol. In some embodiments, the polyol solvent is a polyether polyol. In some embodiments, the polyol solvent is a polyester polyol. Skin compatible polyols of interest include, but are not limited to, glycerol (1,2,3-propanetriol); diglycerol; propylene glycol (1,2-propanediol); dipropylene glycol; 1,3-propanediol; butylene glycol (1,3-butanediol); 1,2-butanediol; pentylene glycol (1,2-pentanediol); 1,5-pentanediol; 1,2-hexanediol; 1,6-hexanediol; 1,2,3-hexanetriol, 1,2,6-hexanetriol; ethoxydiglycol; and dimethyl isosorbide. In some embodiments, the non-aqueous solvent is a glycol ether, a dimethyl ether, or a combination thereof. A non-aqueous solvent is 1,3-propanediol, commercially available from DuPont Tate & Lyle BioProducts LLC under the tradename ZEMEAR. In some embodiments, the non-aqueous solvent is a mixture of 1,3 propanediol and 1,2 hexanediol.

In some embodiments, the subject composition includes about 1 to 50% by weight (e.g. about 1% or more, about 5% or more about 10% or more, about 15% or more, about 20% or more, about 25% or more, about 30% or more, about 35% or more, about 40% or more, about 45% or more, or about 50% or more) of a non-aqueous solvent. In some embodiments, the subject composition includes about 1 to 30% by weight of an agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 50% polyol. In certain embodiments, the agent is glycerin.

In some embodiments, the subject composition includes about 1 to 30% by weight of an agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 99% polyol and one or more additional skin compatible solvents.

### 5.4. Acidifying Agents

In some embodiments, the composition of the present disclosure further comprises one or more acidifying agents. In certain embodiments, the composition comprises 5% to 15% by weight of one or more acidifying agents.

In some embodiments, the one or more acidifying agents is selected from one or more of the group consisting of: gluconolactone, salicylic acid, and a phytic acid solution. In certain embodiments, the one or more acidifying agents is a combination of gluconolactone, salicylic acid, and a phytic acid solution.

In some embodiments, the one or more acidifying agents comprises 5-20% by weight of gluconolactone (e.g. about 5% or more, about 6% or more, about 7% or more, about 8% or more, about 9% or more, about 10% or more, about 11% or more, about 12% or more, about 13% or more, about 14% or more, about 15% or more, about 16% or more, about 17% or more, about 18% or more, about 19% or more, or about 20% or more) of gluconolactone.

In some embodiments, the one or more acidifying agents comprises 0.001 to 3% by weight of salicylic acid. In some embodiments, the composition comprises 0.01% or more, 0.02% or more, 0.03% or more, 0.04% or more, 0.05% or more, 0.06% or more, 0.07% or more, 0.08% or more, 0.09% or more, 0.1% or more, 0.2% or more, 0.3% or more, 0.4% or more, 0.5% or more, 0.6% or more, 0.7% or more, 0.8% or more, 0.9% or more, 1% or more, 2% or more, or 3% of salicylic acid.

In some embodiments, the one or more acidifying agents comprises 0.001 to 3% by weight of a phytic acid solution. In some embodiments, the composition comprises 0.01% or more, 0.02% or more, 0.03% or more, 0.04% or more, 0.05% or more, 0.06% or more, 0.07% or more, 0.08% or more, 0.09% or more, 0.1% or more, 0.2% or more, 0.3% or more, 0.4% or more, 0.5% or more, 0.6% or more, 0.7% or more, 0.8% or more, 0.9% or more, 1% or more, 2% or more, or 3% of a phytic acid solution.

### 5.5. Additional Components

In some embodiments, the composition further comprises one or more additional components.

In some embodiments, the one or more additional components is selected from the group consisting of: Tranexamic Acid, Madecassoside, Pinus Pinaster Bark Extract in Diglycerin, Caprylhydroxamic acid, 1,2-Hexanediol and Propanediol.

In some embodiments, the one or more additional components comprise 0.1% to 8% by weight of Tranexamic (e.g., 0.1% or more, 0.2% or more, 0.3% or more, 0.4% or more, 0.5% or more, 1% or more, 1.5% or more, 2% or more, 2.5% or more, or 3% or more) of Tranexamic.

In some embodiments, the one or more additional components comprise 0.1% to 8% by weight of Madecassoside (e.g., 0.1% or more, 0.2% or more, 0.3% or more, 0.4% or more, 0.5% or more, 1% or more, 1.5% or more, 2% or more, 2.5% or more, or 3% or more) of Madecassoside.

In some embodiments, the one or more additional components comprise 0.1% to 8% by weight of Pinus Pinaster Bark Extract in Diglycerin (e.g., 0.1% or more, 0.2% or more, 0.3% or more, 0.4% or more, 0.5% or more, 1% or more, 1.5% or more, 2% or more, 2.5% or more, or 3% or more) of Pinus Pinaster Bark Extract in Diglycerin.

In some embodiments, the one or more additional components comprise 0.1% to 8% by weight of a mixture of Caprylhydroxamic acid, 1,2-Hexanediol and Propanediol (e.g., 0.1% or more, 0.2% or more, 0.3% or more, 0.4% or more, 0.5% or more, 1% or more, 1.5% or more, 2% or more, 2.5% or more, or 3% or more) of a mixture of Caprylhydroxamic acid, 1,2-Hexanediol and Propanediol.

In some embodiments, the composition comprises 3% by weight of Tranexamic Acid, 0.2% by weight of Madecassoside, 0.5% by weight of Pinus Pinaster Bark Extract in Diglycerin, and a mixture of 1% by weight of Caprylhydroxamic acid, 1,2-Hexanediol and Propanediol.

### 5.6. pH of the Composition

The compositions of the present disclosure comprise a pH ranging from 2.5 to 7. In some embodiments, the composition comprises a pH ranging from 4-6.5. In some embodiments, the composition comprises a pH rangin from 5-6.5. In some embodiments, the composition comprises a pH ranging from 4 to 7. In certain embodiments, the pH is 2.5, 3, 3.5, 4, 4.5 5, 5.5, 6, 6.5 or 7.

### 5.7. Containers

Any containers suitable for storing and/or dispensing the subject formulations can be adapted for use. The container can provide a sealed environment for containing the composition, and separation from the atmosphere. The container can prevent during storage undesirable degradation, e.g., from absorption of light and/or moisture from the atmosphere or surrounding environment. Provided are ready-to-use topical preparations of the composition in a multi-use container which is pre-filled with a storage stable topical composition (e.g., as described herein).

Additional packaging for the container can be included. In some cases, the packaging provides a further barrier that prevents absorption of light and/or moisture from the atmosphere or surrounding environment.

### 5.8. Methods of Preparation

Also provided by this disclosure are processes for storage that include preparation of any one of the subject formulations (e.g., as described herein), e.g., by dissolving niacinamide in an aqueous solvent with a Galactoarabinan and one or more optionally additional components to provide a stable liquid composition capable of storage stability.

In some embodiments, the process includes combining:
1% to 20% by weight niacinamide; and
0.2% to 10% by weight galactoarabinan,
wherein the composition is dissolved in an aqueous solvent and comprises a pH ranging from 2.5 to 7.0.

In certain embodiments, the one or more additional agents are combined and include: 30-40% of a non-aqueous solvent, 10-20% of an acidifying agent, and 1-20% of additional components such as, but not limited to one or more of: Tranexamic Acid, Madecassoside, Pinus Pinaster Bark Extract in Diglycerin, Caprylhydroxamic acid, 1,2-Hexanediol and Propanediol.

Also provided are product storage stable formulations produced by the process according to any one of the embodiments described herein.

### 6. EXAMPLES

### 6.1. Example 1: Exemplary Formulations

The exemplary formulations of Table 1 were prepared and assessed as having desirable properties.

TABLE 1
Components of Exemplary Compositions (% by weight)

| Formulation | 1 (weight %) | 2 | 3 |
|---|---|---|---|
| Niacinamide | 5% Niacinamide | | |
| Galactoarabinan | 2% Galactoarabinan | | |
| Water | 37.71% Water | | |
| Non-Aqueous | 35% Propanediol; | | |
| Solvents | 5% Glycerin | | |
| Acidifying | 10% Gluconolactone; | | |
| Agents | 0.49% Salicylic Acid | | |
| | 0.1% Phytic Acid Solution | | |
| | (50%) | | |
| Additional | 3% Tranexamic Acid; | | |
| Ingredients | 0.2% Madecassoside; | | |
| | 0.5% Pinus Pinaster Bark | | |
| | Extract in Diglycerin (Trade | | |
| | name: Pantrofina Skin 360) | | |
| | 1% Caprylhydroxamic acid, | | |
| | 1,2-Hexanediol and | | |
| | Propanediol (Trade name: | | |
| | Spectrastat PHL) | | |

### 6.2. Example 2: Storage Stability

In order to test storage stability, testing was performed on formulation 2 in table 1. 3 cycles of freeze/thaw testing, and 12 weeks of storage at 5° C., 25° C., 40° C., and 45° C. were performed while observing changes in pH, color, odor, and appearance. Little to no changes were observed in pH and appearance, and slight but acceptable changes were observed in color and odor, demonstrating storage stability.

Initial Stability Readings:

| Color | Odor | Appearance | pH | Viscosity | Sp. Gr. | % Solids |
|---|---|---|---|---|---|---|
| Straw | Characteristic | Clear, non-viscous liquid | 4.04 | N/A | 1.12 | 25.04% |

3 Cycles—Freeze/Thaw

| Cycle | Color | Odor | Appearance | pH | Viscosity |
|---|---|---|---|---|---|
| 1 | Conforms | Conforms | Conforms | 4.09 | N/A |
| 2 | Conforms | Conforms | Conforms | 4.11 | N/A |
| 3 | Conforms | Conforms | Conforms | 4.09 | N/A |
| | | 1 Week | 1 Month | 2 Months | 3 Months |
| | | Date: | | | |
| Temp. | | (Oct. 25, 2021) | (Nov. 18, 2021) | (Dec. 18, 2021) | (Jan. 18, 2022) |
| 5° C. | Color: | Conforms | Conforms | Conforms | Conforms |
| | Odor: | Conforms | Conforms | Conforms | Conforms |
| | Appearance: | Conforms | Conforms | Conforms | Conforms |
| | pH: | 4.13 | 4.09 | 4.10 | 4.12 |
| | Viscosity: | N/A | N/A | N/A | N/A |
| 25° C. | Color: | Conforms* | Conforms* | Conforms* | Conforms* |
| (RT) | Odor: | Conforms | Conforms** | Conforms** | Conforms** |
| | Appearance: | Conforms | Conforms | Conforms | Conforms |
| | pH: | 4.13 | 4.11 | 4.10 | 4.11 |
| | Viscosity: | N/A | N/A | N/A | N/A |
| 40° C. | Color: | Conforms* | Conforms* | Conforms* | Conforms* |
| w/ | Odor: | Conforms | Conforms** | Conforms** | Conforms** |
| 75% RH | Appearance: | Conforms | Conforms | Conforms | Conforms |
| | pH: | 4.15 | 4.12 | 4.13 | 4.15 |
| | Viscosity: | N/A | N/A | N/A | N/A |
| 45° C. | Color: | Conforms* | Conforms* | Conforms* | Conforms* |
| | Odor: | Conforms | Conforms** | Conforms** | Conforms** |
| | Appearance: | Conforms | Conforms | Conforms | Conforms |
| | pH: | 4.14 | 4.13 | 4.14 | 4.14 |
| | Viscosity: | N/A | N/A | N/A | N/A |

Comments:
*The color is slightly darker, but this is acceptable.
**The odor is slightly shifting but this is acceptable.

### 6.3. Example 3: Irritation and Sensitization Potential

Human Repeat Insult Patch Testing (HRIPT) was conducted on Formulation 1 in Table 1 to determine the irritation and sensitization (contact allergy) potential after repeated application to the skin of human subjects, controlling for product dosing and application site, and supervised by a dermatologist.

Human Repeat Insult Patch Testing (HRIPT) was conducted on formulation 1 in table 1 to demonstrate the absence of primary and cumulative irritation potential and skin sensitization potential under maximized conditions, controlling for product dosing and application site, and supervised by a dermatologist. The study duration was 6 weeks and 71 male and female subjects, ranging in age from 18 to 65, completed the study.

### Procedure:

Subjects were requested to bathe or wash as usual before arrival at the facility

Patches containing the test material were then affixed directly to the skin of the intrascapular regions of the back, to the right or left of the midline and subjects were dismissed with instructions not to wet or expose the test area to direct sunlight.

Patches remained in place for 48 hours after the first application. Subjects were instructed not to remove the patches prior to their 48-hour scheduled visit, thereafter, subjects were instructed to remove patches 24 hours after application for the remainder of the study.

This procedure was repeated until a series of eight (8) to nine (9) consecutive, 24-hour exposures had been made three (3) times a week for three (3) consecutive weeks.

Prior to each reapplication, the test sites were evaluated by trained laboratory personnel.

Following a 10-14 day rest period a retest/challenge dose was applied once to a previously unexposed test site. Test sites were evaluated by trained laboratory personnel 48 and 96 hours after application.

In the event of a reaction, the area of erythema and edema were measured. Edema is estimated by the evaluation of the skin with respect to the contour of the unaffected normal skin.

Subjects were instructed to report any delayed reactions that might occur after the final reading.

### Observations:

No adverse reactions of any kind were reported during the course of this study.

There was one (1) subject with a Grade 2 reaction and three (3) subjects with a Grade 1 reaction to the positive control (1.0% Sodium Lauryl Sulfate Solution).

No subjects showed any signs of reaction to the negative control (DI Water).

Conclusions: Under the conditions of the study, there was no indication of a potential to elicit dermal irritation or sensitization (contact allergy) noted for the tested formulation.

### 6.4. Example 3: Clinical Improvements in Signs of Aging

In order to observe the effects on human skin with visible signs of Formulation 1 in Table 1, an independent, 8-week clinical study was conducted with 53 female subjects of diverse ages (37-65) and skin tones (Fitzpatrick types II-V with self-identified African American, Asian, Caucasian, Hispanic, Native American and mixed race individuals). Expert Grading, instrumental measurements, and self-assessments were conducted in order to holistically evaluate the formulation's effects on skin hydration, texture, tone, and visible signs of aging.

### Results:

Immediately after Use:

91% of users agreed skin looked and felt more hydrated

Researchers observed a statistically significant improvement in skin's moisture levels*(p<0.001) and radiance**(p<0.001)

### After 4 Weeks:

94% of users agreed that skin texture was visibly smoother. 84% of users agreed skin looked more youthful and healthy.

Researchers observed a statistically significant improvement in skin tone evenness and brightness**(p<0.001).

Measurements and analysis confirmed statistically significant improvements in skin texture***(p<0.001).

### After 8 Weeks:

90% of users agreed that skin looked firmer and more elastic.

90% of users agreed that there was a visible improvement in skin clarity.

83% of users agreed that pores appeared smaller and less visible.

Researchers observed statistically significant improvements in fine lines and wrinkles**(p=0.002) and long-term skin hydration*(p<0.001).

Measurements and analysis confirmed statistically significant improvements in skin texture***(p=0.004).

*as measured by Corneometer CM 825 instrumentation.

**as measured by expert grading of photographs captured by a VISIA-CR device.

***as measured and analyzed by VisioScan VC 20 instrumentation.

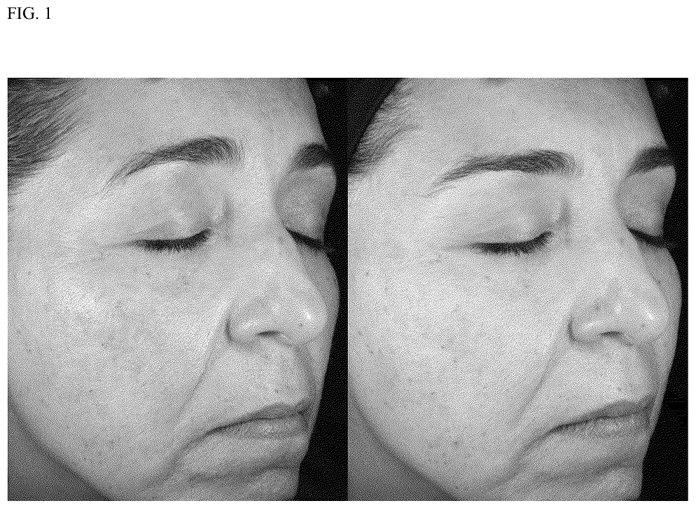

Representative photography of before application of the formulation, and 8 weeks after application of the formulation is shown in FIG. 1. FIG. 1 provides Baseline (left photo) photography taken via Visia-CR device and shows fine wrinkling in the periocular region, as well as hyperpigmentation and uneven skin tone across the cheeks and forehead. After 8 weeks (right photo), fine wrinkling, uneven skin tone, and hyperpigmentation are visibly improved.

### 7. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A storage stable, topical composition comprising:
a. 1% to 20% by weight niacinamide; and
b. 0.2% to 10% by weight galactoarabinan,
wherein the composition is dissolved in an aqueous solvent and comprises a pH ranging from 2.5 to 7.0.

2. The storage stable, topical composition consists essentially of:
a. 1% to 20% by weight niacinamide; and
b. 0.2% to 10% by weight galactoarabinan,
wherein the composition is dissolved in an aqueous solvent and comprises a pH ranging from 2.5 to 7.0.

3. The storage stable, topical composition of claim 1, wherein the composition does not comprise N-Acetylglucosamine (GlcNAc).

4. The storage stable, topical composition of any one of claims 1-3, wherein the pH ranges from 4 to 7.

5. The storage stable, topical composition of any one of claims 1-4, wherein the composition comprises 5% Niacinamide.

6. The storage stable, topical composition of any one of claims 1-5, wherein the composition comprises 2% Galactoarabinan.

7. The storage stable, topical composition of any one of claims 1-6, wherein the composition comprises 30%-40% of the aqueous solvent.

8. The storage stable, topical composition of any one of claims 1-7, wherein the composition comprises 35%-40% of the aqueous solvent.

9. The storage stable, topical composition of any one of claims 1-8, wherein the aqueous solvent is water.

10. The storage stable, topical composition of any one of claims 1-9, wherein the composition further comprises one or more non-aqueous solvents.

11. The storage stable, topical composition of claim 10, wherein the one or more non-aqueous solvents is selected from the group consisting of: 1,3 propanediol, 1,2 propanediol, 1,3 butanediol, 1,5 pentanediol, 1,2 hexanediol, 1,6 hexanediol, glycerol, diglycerol, ethoxydiglycol, and dimethyl isosorbide.

12. The storage stable, topical composition of any one of claims 1-11, wherein the one or more non-aqueous solvents is propanediol.

13. The storage stable, topical composition of any one of claims 1-11, wherein the one or more non-aqueous solvent is a mixture of propanediol and glycerin.

14. The storage stable, topical composition of any one of claims 1-13, wherein the composition further comprises 5% to 15% by weight of one or more acidifying agents.

15. The storage stable, topical composition of claim 14, wherein the one or more acidifying agents is selected from the group consisting of: gluconolactone, salicylic acid, and a phytic acid solution.

16. The storage stable, topical composition of any one of claims 14-15, wherein the one or more acidifying agents comprises 5-20% by weight of gluconolactone.

17. The storage stable, topical composition of any one of claims 14-16, wherein the one or more acidifying agents comprises 0.001 to 3% of salicylic acid.

18. The storage stable, topical composition of any one of claims 14-17, wherein the one or more acidifying agents comprises 0.001 to 3% of and a phytic acid solution.

19. The storage stable, topical composition of any one of claims 1-16, wherein the composition further comprises one or more additional components.

20. The storage stable, topical composition of claim 17, wherein the one or more additional components is selected from the group consisting of: Tranexamic Acid, Madecassoside, Pinus Pinaster Bark Extract in Diglycerin, Caprylhydroxamic acid, 1,2-Hexanediol and Propanediol.

21. The storage stable, topical composition of claim 18, wherein the composition comprises 3% Tranexamic Acid, 0.2% Madecassoside, 0.5% Pinus Pinaster Bark Extract in Diglycerin, and a mixture of 1% Caprylhydroxamic acid, 1,2-Hexanediol and Propanediol.

22. The storage stable, topical composition of any one of claims 1-21, wherein the composition is in the form of a serum, lotion, or cream.

23. A ready-to-use topical preparation in a multi-use container which is pre-filled with a storage stable topical composition according to any one of claims 1-22, wherein the multi-use container comprises means for dispensing a single dose of the storage stable topical composition.

24. The preparation of claim 23, wherein the storage stable topical composition is sealed from the atmosphere in the container.

25. The preparation of any one of claims 22-24, wherein the container is placed in packaging.
